(19) United States
(12) Patent Application Publication
(10) Pub. No.: US 2024/0299051 A1
(43) Pub. Date: Sep. 12, 2024

(54) INTRAVASCULAR LITHOTRIPSY BALLOON PERFUSION CATHETERS

(71) Applicant: Shockwave Medical, Inc., Santa Clara, CA (US)

(72) Inventors: Devinder Kaur SIDHU, Santa Clara, CA (US); Rainier BETELIA, San Jose, CA (US); Todd JENKINS, San Jose, CA (US); Danielle ROND, Santa Clara, CA (US)

(73) Assignee: Shockwave Medical, Inc., Santa Clara, CA (US)

(21) Appl. No.: 18/593,691

(22) Filed: Mar. 1, 2024

Related U.S. Application Data
(60) Provisional application No. 63/451,115, filed on Mar. 9, 2023.

(51) Int. Cl. A61B 17/22; A61B 17/00; A61M 25/10

(52) U.S. Cl. CPC A61B 17/22022; A61B 17/00234; A61B 2017/00292; A61B 2017/22025; A61M 25/1002; A61M 25/1011; A61M 2025/1086

(57) ABSTRACT

The present disclosure provides a balloon catheter for treating a stenosis in a body lumen that incorporates at least one shock wave generator located within at least one balloon and at least one channel that permits blood to flow through the body lumen past the balloon while the balloon is inflated. The at least one channel acts as a bypass around the inflated balloon(s) of the catheter such that blood flow through the lumen is generally not fully blocked during a shock wave treatment.

### CROSS-REFERENCE TO RELATED APPLICATIONS

This application claims priority to U.S. Provisional Patent Application Ser. No. 63/451,115, entitled INTRAVASCULAR LITHOTRIPSY BALLOON PERFUSION CATHETERS, filed Mar. 9, 2023, the entire contents of which is hereby incorporated by reference.

### FIELD OF THE DISCLOSURE

The present disclosure relates generally to the field of medical devices and methods, and more specifically to shock wave catheter devices for treating calcified lesions in body lumens, such as calcified lesions and occlusions in vasculature and kidney stones in the urinary system.

### BACKGROUND

A wide variety of catheters have been developed for treating calcified lesions, such as calcified lesions in vasculature associated with arterial disease. For example, treatment systems for percutaneous coronary angioplasty or peripheral angioplasty use angioplasty balloons to dilate a calcified lesion and restore normal blood flow in a vessel. In these types of procedures, a catheter carrying a balloon is advanced into the vasculature along a guide wire until the balloon is aligned with calcified plaques. The balloon is then pressurized (normally to greater than 10 atm), causing the balloon to expand in the vessel to push calcified plaques back into the vessel wall and dilate occluded regions of vasculature.

When relying on an angioplasty balloon to dilate occluded regions of vasculature, one concern is the temporary blockage of blood flow while the balloon is inflated. Lack of blood flow within a vessel (e.g., ischemia) can lead to hypoxia and microvascular dysfunction, which may lead to heart attack, organ failure, or stroke. To prevent ischemia, the treatment period during which an angioplasty balloon is inflated can be reduced, such as by limiting the inflation period to 60 seconds. The treatment protocol may also involve alternating periods where the balloon is inflated with periods where the balloon is not inflated during a procedure to allow blood to flow past the device intermittently. Limiting the treatment to only short and/or intermittent periods, however, extends the total treatment period and may introduce other issues such as increased wear on components of the device from repeatedly inflating and deflating the balloon multiple times within a single procedure.

Although limited treatment periods may be effective in some applications, limited treatment periods can be ineffective for certain treatment protocols. For instance, when treating calcification in the stenotic valve leaflets of the heart ventricle (balloon valvuloplasty), the valve heart orifice cannot be blocked by a balloon for more than about 10-15 seconds. During balloon valvuloplasty, physicians typically remove the catheter after a single cycle of inflating the balloon and do not repeat the inflation process, optionally placing prosthesis in place of the heart valve if heart function is still unsatisfactory. If the pre-treatment of calcification before placing the prosthesis was not sufficient, that may lead to paravalvular leaks and uneven opening or placement of prosthesis. Thus, during balloon valvuloplasty, limited treatment periods and/or intermittent periods of inflation may not be sufficient to treat calcification in the heart ventricle.

More recently, the technique and treatment of intravascular lithotripsy (IVL) has been developed, which is an interventional procedure to modify calcified plaque in diseased arteries. The mechanism of plaque modification is through use of a catheter having one or more acoustic shock wave generating sources located within a liquid that can generate acoustic shock waves that modify the calcified plaque. IVL devices vary in design with respect to the energy source used to generate the acoustic shock waves, with two exemplary energy sources being electrohydraulic generation and laser generation.

For electrohydraulic generation of acoustic shock waves, a conductive solution (e.g., saline) may be contained within an enclosure that surrounds electrodes or can be flushed through a tube that surrounds the electrodes. The calcified plaque modification is achieved by creating acoustic shock waves within the catheter by an electrical discharge across the electrodes. The energy from this electrical discharge enters the surrounding fluid faster than the speed of sound, generating an acoustic shock wave. In addition, the energy creates one or more rapidly expanding and collapsing vapor bubbles that generate secondary shock waves. The shock waves propagate radially outward and modify calcified plaque within the blood vessels. For laser generation of acoustic shock waves, a laser pulse is transmitted into and absorbed by a fluid within the catheter. This absorption process rapidly heats and vaporizes the fluid, thereby generating the rapidly expanding and collapsing vapor bubble, as well as the acoustic shock waves that propagate outward and modify the calcified plaque. The acoustic shock wave intensity is higher if a fluid is chosen that exhibits strong absorption at the laser wavelength that is employed. These examples of IVL devices are not intended to be a comprehensive list of potential energy sources to create IVL shock waves.

The IVL process may be considered different from standard atherectomy procedures in that it cracks calcium but does not liberate the cracked calcium from the tissue. Hence, generally speaking, IVL should not require aspiration nor embolic protection. Further, due to the compliance of a normal blood vessel and non-calcified plaque, the shock waves produced by IVL do not modify the normal vessel tissue or non-calcified plaque. Moreover, IVL does not carry the same degree of risk of perforation, dissection, or other damage to vasculature as atherectomy procedures or angioplasty procedures using cutting or scoring balloons.

More specifically, catheters to deliver IVL therapy have been developed that include pairs of electrodes for electrohydraulically generating shock waves inside an angioplasty balloon. Shock wave devices can be particularly effective for treating calcified plaque lesions because the acoustic pressure from the shock waves can crack and disrupt lesions near the angioplasty balloon without harming the surrounding tissue. In these devices, the catheter is advanced over a guidewire through a patient's vasculature until it is positioned proximal to and/or aligned with a calcified plaque lesion in a body lumen. The balloon is then inflated with conductive fluid (using a relatively low pressure of 2-4 atm) so that the balloon expands to contact the lesion but is not an inflation pressure that substantively displaces the lesion. Voltage pulses can then be applied across the electrodes of the electrode pairs to produce acoustic shock waves that propagate through the walls of the angioplasty balloon and into the lesions. Once the lesions have been cracked by the acoustic shock waves, the balloon can be expanded further to increase the cross-sectional area of the lumen and improve blood flow through the lumen. Alternative devices to deliver IVL therapy can be within a closed volume other than an angioplasty balloon, such as a cap, balloons of variable compliancy, or other enclosure. Despite these advances, currently available shock wave catheters may also encounter issues with ischemia by restricting blood flow while the balloon is inflated during treatment.

### BRIEF SUMMARY

According to an aspect, a catheter includes a shock wave generator surrounded by a balloon and at least one channel that permits blood to flow past the balloon while the balloon is inflated. Unlike conventional angioplasty catheters that rely on contact with a pressurized balloon to push back plaque, the catheters described herein can better utilize multiple balloon configurations because the shock wave devices generate shock waves that radiate outward to break up occlusions, which may remove the need for repeatedly deflating, rotating, and inflating the shock wave catheter to effectively reduce the plaque in a body lumen. Moreover, while providing a channel to permit blood flow, the catheters described herein realize improved calcification reduction even in single balloon configurations, as the shock waves can crack the occlusions while the balloon is inflated.

According to an aspect, a catheter for treating a stenosis in a body lumen can comprise: an elongated tube, at least one shock wave generator comprising at least one electrode pair, at least one balloon sealed to a distal end of the elongated tube and surrounding the at least one shock wave generator, the at least one balloon fillable with a conductive fluid, and at least one channel that permits blood to flow through the body lumen past the at least one balloon while the at least one balloon is inflated.

The at least one channel may permit blood to flow through the body lumen at a flow rate that is at least 50% of a normal flow rate of blood through the body lumen without the catheter positioned in the body lumen. The at least one channel may be defined by at least one lumen that extends externally to the elongated tube. The at least one lumen can extend outside of an outer surface of the at least one balloon. The at least one channel can extend within the elongated tube. The elongated tube can comprise at least one opening to the at least one channel that is located proximally of the at least one balloon for blood to flow into or out of the at least one channel, and at least one opening to the at least one channel from the at least one channel that is located distally of the at least one balloon for blood to flow into or out of the at least one channel.

The catheter may comprise a plurality of balloons and a plurality of shock wave generators, each balloon of the plurality of balloons being sealed to a region of the elongated tube and surrounding one or more of the plurality of shock wave generators, and the at least one channel can be defined by separation between the plurality of balloons. The plurality of balloons can be at least three balloons. A cross section of each of the at least three balloons can comprise a circular shape. A cross section of each of the at least three balloons can comprise an elliptical shape. The at least one balloon may comprise a plurality of lobes that extend outwardly from the elongated tube, and the at least one channel may be defined by separation between the plurality of lobes of the at least one balloon. The at least one balloon may comprise a crescent shape when inflated, and the at least one channel is defined by space between the at least one balloon and the body lumen. The at least one balloon can comprise a double crescent shape when inflated, and the at least one channel can be defined by space between the at least one balloon and the body lumen. The elongated tube may comprise a guidewire lumen for receiving a guidewire, and the catheter can be configured to be advanced into the body lumen over the guidewire.

According to an aspect, a system for treating a stenosis in a body lumen can comprise: a catheter comprising: an elongated tube, at least one shock wave generator comprising at least one electrode pair, at least one balloon sealed to a distal end of the elongated tube and surrounding the at least one shock wave generator, the at least one balloon fillable with a conductive fluid, and at least one channel that permits blood to flow through the body lumen past the at least one balloon while the at least one balloon is inflated, and a power source configured to apply a voltage pulse to the at least one shock wave generator to generate shock waves for treating the stenosis.

The at least one channel may permit blood to flow through the body lumen at a flow rate that is at least 50% of a normal flow rate of blood through the body lumen without the catheter positioned in the body lumen. The at least one channel can be defined by at least one lumen that extends externally to the elongated tube. The at least one lumen can extend outside of an outer surface of the at least one balloon. The at least one channel can extend within the elongated tube. The elongated tube can comprise at least one opening to the at least one channel that is located proximally of the at least one balloon for blood to flow into or out of the at least one channel, and at least one opening to the at least one channel from the at least one channel that is located distally of the at least one balloon for blood to flow into or out of the at least one channel.

The catheter may comprise a plurality of balloons and a plurality of shock wave generators, each balloon of the plurality of balloons being sealed to a region of the elongated tube and surrounding one or more of the plurality of shock wave generators, and the at least one channel can extend between the plurality of balloons. The plurality of balloons can be at least three balloons. A cross section of each of the at least three balloons can comprise a circular shape. A cross section of each of the at least three balloons can comprise a circular shape. The at least one balloon may comprise a plurality of lobes that extend outwardly from the elongated tube, and wherein the at least one channel is defined by separation between the plurality of lobes of the at least one balloon. The at least one balloon can comprise a crescent shape when inflated, and wherein the at least one channel is defined by separation between the plurality of lobes of the at least one balloon. The at least one balloon can comprise a double crescent shape when inflated, and wherein the at least one channel is defined by separation between the plurality of lobes of the at least one balloon. The elongated tube can comprise a guidewire lumen for receiving a guidewire, and wherein the catheter is configured to be advanced into the body lumen over the guidewire.

According to an aspect, a method for treating a stenosis in a body lumen can comprise: advancing a catheter within the body lumen to a position proximate to the stenosis, inflating at least one balloon of the catheter so that an outer surface of the at least one balloon contacts the body lumen and blood is permitted to flow through the body lumen past the at least one balloon while the at least one balloon is inflated, and generating shock waves via at least one shock wave generator of the catheter while blood is flowing through the body lumen past the at least one balloon.

The method may comprise advancing a guidewire within the body lumen to locate the stenosis and advancing the catheter over the guidewire. The method may comprise retracting the guidewire before inflating the balloon. The method may comprise retracting the guidewire such that a distal end of the guidewire is located proximally of a proximal end of the balloon. Retracting the guidewire can comprise withdrawing the guidewire from the body lumen.

### BRIEF DESCRIPTION OF THE FIGURES

Illustrative aspects of the present disclosure are described in detail below with reference to the following drawing figures. It is intended that the embodiments and figures disclosed herein are to be considered illustrative rather than restrictive.

FIG. 1 illustrates a system comprising an exemplary shock wave catheter being used to treat a stenosis in a blood body lumen, according to one or more aspects of the present disclosure.

FIG. 2A illustrates the distal end of an exemplary shock wave perfusion balloon catheter comprising an internal channel, according to one or more aspects of the present disclosure.

FIG. 2B illustrates a cross sectional view of an exemplary elongated tube comprising an internal channel that is usable for a shock wave perfusion balloon catheter, according to one or more aspects of the present disclosure.

FIG. 2C illustrates a cross sectional view of an exemplary elongated tube comprising a central guidewire lumen and an internal channel that is usable for a shock wave perfusion balloon catheter, according to one or more aspects of the present disclosure.

FIG. 3A illustrates the distal end of an exemplary shock wave perfusion balloon catheter that has at least one external lumen that is usable for a shock wave perfusion balloon catheter, according to one or more aspects of the present disclosure.

FIG. 3B illustrates a cross sectional view of the distal end of the shock wave perfusion balloon catheter of FIG. 3A.

FIG. 4A illustrates the distal end of an exemplary tri-lobe balloon shock wave catheter, according to one or more aspects of the present disclosure.

FIG. 4B illustrates a cross sectional view of a tri-lobe balloon shock wave catheter comprising circular balloons, according to one or more aspects of the present disclosure.

FIG. 4C illustrates a cross sectional view of a tri-lobe balloon shock wave catheter comprising elliptical balloons, according to one or more aspects of the present disclosure.

FIG. 5 illustrates the distal end of an exemplary tri-lobe balloon shock wave catheter comprising a central elongated tube, according to one or more aspects of the present disclosure.

FIG. 6A illustrates the distal end of a crescent-shaped balloon shock wave catheter, according to one or more aspects of the present disclosure.

FIG. 6B illustrates a cross sectional view of the catheter of FIG. 6A positioned within a body lumen, according to one or more aspects of the present disclosure.

FIG. 7A illustrates the distal end of a double crescent-shaped balloon shock wave catheter, according to one or more aspects of the present disclosure.

FIG. 7B illustrates a cross sectional view of the catheter of FIG. 7A positioned within a body lumen, according to one or more aspects of the present disclosure.

### DETAILED DESCRIPTION

The following description is presented to enable a person of ordinary skill in the art to make and use the various embodiments and aspects thereof disclosed herein. Descriptions of specific devices, assemblies, techniques, and applications are provided only as examples. Various modifications to the examples described herein will be readily apparent to those of ordinary skill in the art, and the general principles described herein may be applied to other examples and applications without departing from the spirit and scope of the various embodiments and aspects thereof. Thus, the various embodiments and aspects thereof are not intended to be limited to the examples described herein and shown, but are to be accorded the scope consistent with the claims.

Described herein are balloon catheters incorporating at least one channel that permits blood to flow past the balloon while the balloon is inflated. When the catheter is positioned in a body lumen (such as a vessel or valve), the channel acts as a bypass around the inflated balloon of the catheter such that blood flow through the body lumen is generally not fully blocked during treatment.

The at least one channel may be provided in a number of different ways. In some examples, a channel extends within an elongated tube that the balloon of the catheter is sealed to. In some examples, a channel is provided by an external lumen that extends externally of the balloon. In some examples, at least one channel is provided by the configuration of one or more balloons. For example, the catheter may include a plurality of balloons that are spaced apart from one another such that at least one channel is formed by the spacing between the balloons. The catheter may include a single balloon comprising a plurality of lobes, such as a tri-lobe balloon comprising lobes that extend outwardly from an elongated tube that the tri-lobe balloon is sealed to. In such configuration, the channel (or channels) is defined by the space between the lobes of the tri-lobe balloon. A tri-lobe balloon can have multiple circular and/or elliptical balloons. The catheter may include a balloon shaped to not occupy the entire body lumen when inflated, leaving space for blood to flow past the balloon. For example, the balloon could have a crescent shape that allows blood to flow in the space within the body lumen that is opposite the balloon.

The catheter designs described include at least shock wave generator that comprises at least one electrode pair within the working length of a catheter that delivers acoustic shock waves and/or cavitation bubbles to a treatment site proximate to the catheter's distal tip. For instance, as described in U.S. Pat. No. 10,709,462, incorporated herein by reference in its entirety, a first electrode of a shock wave generator can be formed from a side edge of a conductive metal sheath mounted within the catheter. An electrode pair can be formed by positioning a second conductive material a controlled distance (i.e., a gap, also referred to as a “spark gap”) apart from the conductive sheath to allow for a reproducible arc across the electrodes for a given current and voltage. In some examples, as described in the above reference, a second electrode of an electrode pair can be formed by from an electrically conductive portion (e.g., an insulation-removed or non-insulated portion) of a wire extending along the length of the catheter. Additionally or alternatively, as described herein, an exemplary electrode pair can be formed from two cylindrical conductive metal sheaths mounted concentrically within a catheter. Such an electrode assembly may have a relatively smaller crossing profile compared to existing electrode assembly designs, for instance, with a crossing profile between 0.8 mm to 1.2 mm in diameter. Such an electrode assembly design may also facilitate manufacturing of a catheter by simplifying the process for constructing the electrode assembly.

Efforts have been made to improve the design of electrode assemblies included in shock wave and directed cavitation catheters. For instance, low-profile electrode assemblies have been developed that reduce the crossing profile of a catheter and allow the catheter to more easily navigate calcified vessels to deliver shock waves in more severely occluded regions of vasculature. Examples of low-profile electrode designs that can be used in any of the embodiments described herein can be found in U.S. Pat. Nos. 8,888,788, 9,433,428, and 10,709,462 (mentioned above), and in U.S. Publication No. 2021/0085383 all of which are incorporated herein by reference in their entireties. Other catheter designs have improved the delivery of shock waves, for instance, by specific electrode construction and configuration thereby directing shock waves in a forward direction to break up tighter and harder-to-cross occlusions in vasculature. Examples of forward-firing catheter designs that can be used for the catheter designs described herein can be found in U.S. Pat. Nos. 10,966,737, 11,478,261, and 11,596,423 and U.S. Publication Nos. 2023/0107690 and 2023/0165598, all of which are incorporated herein by reference in their entireties.

The catheter designs described herein, according to various examples, can be configured such that the flow rate of the blood that flows through the one or more channels of the catheter when the balloon is fully inflated is at least 50% of the flow rate of blood that would normally flow through the body lumen without the catheter placed in the body lumen (i.e., the “normal flow rate”). In one or more examples, the flow rate of blood that flows through the channel(s) of the catheter may be less than 50% of the normal flow rate, such as 10%, 15%, 20%, or 25% of the normal flow rate. In one or more examples, the flow rate of blood that flows through the channel(s) of the catheter may be more than 50% of the normal flow rate, such as 70%, 80%, or 90% of the normal flow rate. It should be understood that the exemplary ranges for blood flow rate described in this disclosure include increments and gradients of percentage within and about the expressly disclosed ranges. As used herein, “fully inflated” can mean that the balloon has been inflated until it contacts the body lumen and/or an occluded region of the body lumen during normal operation of the catheter (e.g., when shockwaves are being used to treat an occlusion) or has been inflated to a maximum amount that the balloon is configured to inflate during normal operation (e.g., when shockwaves are being used to treat an occlusion).

In some embodiments, the blood flow rate through a body lumen when a catheter's balloon(s) are inflated is proportional to a total cross-sectional area of the catheter's one or more channels relative to a total cross-sectional area of the catheter. For example, a catheter whose channel(s) permit 10% or less of normal blood flow rate through a body lumen when the balloon(s) are inflated, may have a total channel cross-sectional area that is 10% or less of the total cross-sectional area of the catheter.

As used herein, the term “electrode” refers to an electrically conducting element (typically made of metal) that receives electrical current and subsequently releases the electrical current to another electrically conducting element. In the context of the present disclosure, electrodes are often positioned relative to each other, such as in an arrangement of an inner electrode and an outer electrode. Accordingly, as used herein, the term “electrode pair” refers to two electrodes that are positioned adjacent to each other such that application of a sufficiently high voltage to the electrode pair will cause an electrical current to transmit across the gap (also referred to as a “spark gap”) between the two electrodes (e.g., from an inner electrode to an outer electrode, or vice versa, optionally with the electricity passing through a conductive fluid or gas therebetween). In some contexts, one or more electrode pairs may also be referred to as an electrode assembly. In the context of the present disclosure, the term “emitter” broadly refers to the region of an electrode assembly where the current transmits across the electrode pair, generating a shock wave. Emitters can be singular, paired, or otherwise arranged together to be electrically connected as an emitter assembly. Shock waves can be generated at each electrode pair of an emitter; accordingly emitters can also be referred to as “shock wave generators”.

In some embodiments, an IVL catheter is a so-called “rapid exchange-type” (“Rx”) catheter provided with an opening portion through which a guide wire is guided (e.g., through a middle portion of a central tube in a longitudinal direction). In other embodiments, an IVL catheter may be an “over-the-wire-type” (“OTW”) catheter in which a guide wire lumen is formed throughout the overall length of the catheter, and a guide wire is guided through the proximal end of a hub.

Although shock wave devices described herein generate shock waves based on high voltage applied to electrodes, it should be understood that a shock wave device additionally or alternatively may comprise a laser and optical fibers as a shock wave emitter system whereby the laser source delivers energy through an optical fiber and into a fluid to form shock waves and/or cavitation bubbles.

In the following description of the various embodiments, reference is made to the accompanying drawings, in which are shown, by way of illustration, specific embodiments that can be practiced. It is to be understood that other embodiments and examples can be practiced, and changes can be made without departing from the scope of the disclosure.

In addition, it is also to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof. As provided herein, it should be appreciated that any disclosure of a numerical range describing dimensions or measurements such as thicknesses, length, weight, time, frequency, temperature, voltage, current, angle, etc. is inclusive of any numerical increment or gradient within the ranges set forth relative to the given dimension or measurement.

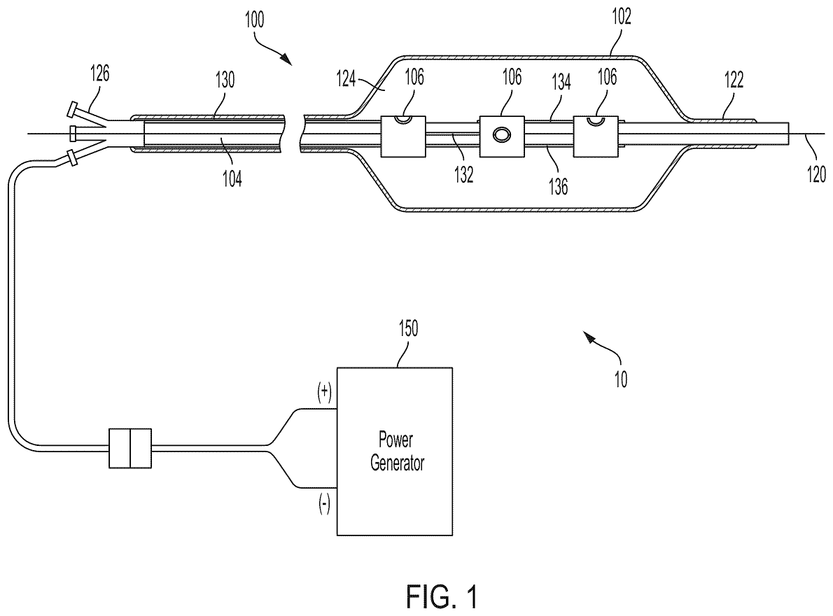

FIG. 1 depicts an exemplary system 10 comprising a shock wave catheter 100 according to one or more examples of the disclosure. The shock wave catheter 100 includes an elongated tube 104 and a balloon 102. The balloon 102 wraps circumferentially around a portion of the elongated tube 104 in a sealed configuration via, for example, a seal 122. The balloon 102 forms an annular channel 124 around the elongated tube 104 through which a conductive fluid, such as saline, may be admitted to the balloon 102 via fill ports 126. The balloon 102 is filled with the conductive fluid such that the balloon 102 can be inflated and gently contact the walls of a body lumen (such as the walls of an artery proximate to a calcified lesion). Unlike traditional angioplasty balloons which are often inflated to a pressure where the exterior of such balloons are frictionally fit to the vessel walls, the balloon 102 herein can be inflated to a relatively lower pressure sufficient to apposition the exterior of balloon 102 at a target location within a body lumen, thereby forming gentle contact with the walls of a body lumen. In one or more examples, the conductive fluid may also contain an x-ray contrast fluid to permit fluoroscopic viewing of the catheter by a surgeon during use.

The elongated tube 104 can include a number of longitudinal grooves or channels configured for retaining wires, fiber optic cables, and/or inner electrodes. The elongated tube 104 of FIG. 1 includes four grooves that extend along the length of the elongated tube 104, which receive insulated wires 130, 132, 134, and 136 (which may be fiber optic cables in other embodiments). The distal ends of the insulated wires connect to a number of shock wave generators 106 located within the balloon 102 and circumferentially wrapped around the elongated tube 104. Each of the shock wave generators 106 includes at least one electrode pair, with the electrodes of each pair spaced apart from one another to define a spark gap. The distance between electrodes of an electrode pair may vary according to the magnitude of the high voltage pulse applied to the shock wave generator 106. For example, a gap of about 0.004 inches to about 0.006 inches may be effective for shock wave generation using voltage pulses of about 3,000 V.

The system 10 includes a power source 150 (e.g., a variable high voltage pulse generator, a laser pulse generator, etc.) that is connected to the proximal ends of the insulated wire 130 and the insulated wire 136. The insulated wires provide voltage to the shock wave generators 106. As voltage is applied across the insulated wires by the power source 150, each pulse initially ionizes the conductive fluid inside the balloon 102 to create small gas bubbles around the shock wave generators 106 that insulate the electrodes. Subsequently, a plasma arc forms across a gap between the electrodes of the electrode pairs, creating a low impedance path where current flows freely. The heat from the plasma arc heats the conductive fluid to create a rapidly expanding vapor bubble. The expansion and collapse of the vapor bubble creates a shock wave that radiates outwardly though the annular channel 124 within the balloon 102 and then through the blood to the calcified lesion proximate to the balloon 102.

As shown in FIG. 1, the catheter 100 has three shock wave generators 106; however, this is provided for example only and should not be construed as limiting in any manner as the catheter 100 could include one shock wave generator, two shock wave generators, or more than three shock wave generators. When the catheter 100 includes multiple shock wave generators, the shock wave generators may be located closely longitudinally adjacent to one another such that the shock waves generators can constructively interfere with one another, as described in U.S. application Ser. No. 16/967,544, which is hereby incorporated by reference. For instance, the shock wave generators 106 can be spaced apart longitudinally less than 6 mm from one another, such as spaced apart by a distance between 1 mm and 4 mm (or at increments of distance therebetween), such that the shock waves generated at a first shock wave generator and a second shock wave generator constructively interfere to produce a combined shock wave.

Moreover, as described in the above reference, a shock wave generator can include multiple electrode pairs that are configured to constructively interfere with one another. For instance, a single shock wave generator can include multiple electrode pairs that are located at essentially the same longitudinal location as one another but are circumferentially offset from one another by an angle of less than 180 degrees, such as an angle between 40 and 140 degrees, an angle between 65 and 125 degrees, or an angle between 80 and 100 degrees, such that shock waves generated at the first electrode pair and the second electrode pair constructively interfere to produce a combined shock wave.

The elongated tube 104 includes a lumen through which a guidewire 120 is inserted. In operation, a physician uses the guidewire 120 to guide the elongated tube 104 into position proximate to a calcified lesion in a body lumen. Once positioned, the power source 150 is used to deliver a series of pulses to create a series of shock waves at the shock wave generators 106 within the balloon 102 and within the body lumen being treated. The magnitude of the shock waves can be controlled by controlling the magnitude of the pulsed voltage, the current, the duration, and the repetition rate of the voltage supplied by the power source 150. The physician may start with low energy shock waves and increase the energy as needed to crack calcified plaques. Such shock waves will be conducted through the conductive fluid within the balloon 102, through the blood to the calcified lesion where the energy will break apart or crack the hardened plaque.

In one or more examples, the system 10 can be configured to generate shock waves via laser power. The power generator 150 can be a laser pulse generator that delivers laser pulses to the shock wave generators 106 via fiber optics cables rather than wires. In such implementations, the fiber optics may be exposed to the conductive fluid such that the laser pulse is emitted directly into the fluid. When a laser pulse is delivered to the shock wave generators 106, a laser-generated bubble can form (in a similar manner as described with respect to electrical arc shock wave formation) and produce shock waves during the rapid expansion and collapse of the laser-generated bubble.

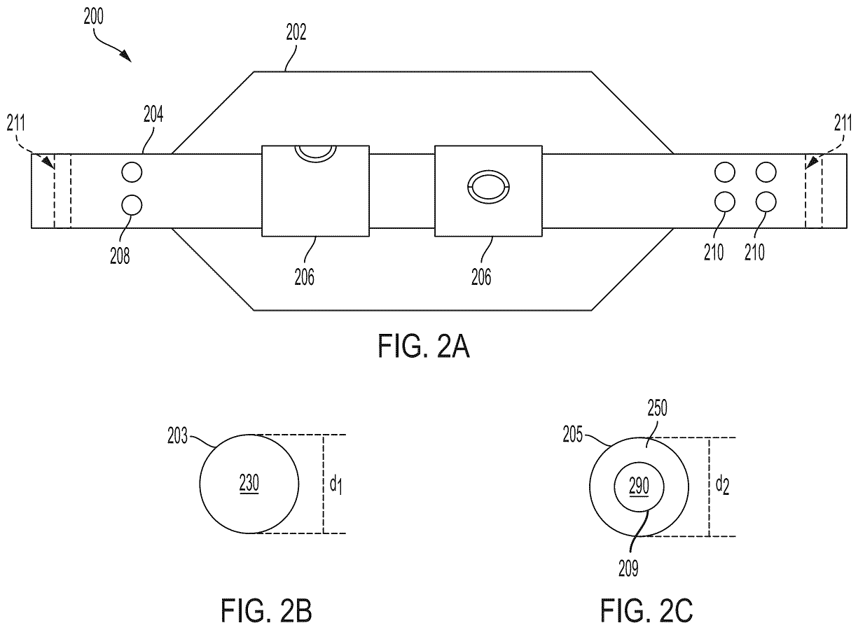

FIG. 2A illustrates the distal end of an exemplary shock wave perfusion balloon catheter 200 comprising an internal channel. The catheter 200 includes an elongated tube 204 and a balloon 202 sealed to the elongated tube 204. The balloon 202 surrounds a pair of shock wave generators 206, which are circumferentially wrapped around the elongated tube 204. In one or more examples, the balloon 202 can surround more than or less than two shock wave generators 206. Each of the shock wave generators 206 includes at least one electrode pair, with the electrodes of each electrode pair spaced apart from one another to form a spark gap. The balloon 202 is fillable with conductive fluid, such as saline, such that the balloon 202 expands (i.e., inflates) to provide an annular channel between an inner surface of the balloon 202 and the shock wave generators 206.

The catheter 200 can be used for catheter 100 in system 10 of FIG. 1 and can be connected to a pulsed power source such as the power source 150 to supply voltage to shock wave generators within the balloon 202 of the catheter 200 to generate shock waves at the spark gaps between the electrodes of the electrode pairs of the shock wave generators 206. The catheter 200 is illustrated with a pair of shock wave generators 206. However, this is provided for explanatory purposes only, and the catheter 200 may a single shock wave generator 206 or more than two shock wave generators 206 within the balloon 202.

The catheter 200 includes an internal channel to permit blood to flow past the balloon 202 while the balloon 202 is inflated. The internal channel extends within the elongated tube 204, which includes at least one proximal hole 208 located proximally of a proximal side of the balloon 202 and at least one distal hole 210 located distally of a distal side of the balloon 202. When the balloon 202 is located in a body lumen and filled with fluid (i.e., when inflated), blood can enter the internal channel via the proximal holes 208, flow through the internal channel, and exit the internal channel via the distal holes 210. If blood is flowing in the opposite direction, blood may enter the internal channel via the distal holes 210 and exit the internal channel via the proximal holes 208. Accordingly, the internal channel provides a channel that permits blood to flow through the body lumen past the balloon 202 while the balloon 202 is inflated. As shown, the elongated tube 204 includes a pair of proximal holes 208, however, the elongated tube 204 may include a single proximal hole 208 or more than two proximal holes 208. Furthermore, the elongated tube 204 may include more or fewer distal holes 210 than the four distal holes 210 illustrated in FIG. 2A.

The flow rate of the blood that flows through the internal channel of the catheter 200 when the balloon 202 is fully inflated can be approximately 50% of the flow rate of blood that would normally flow through the body lumen without the catheter 200 placed in the body lumen (i.e., the “normal flow rate”). In one or more examples, the flow rate of blood that flows through the internal channel of the catheter 200 may be less than 50% of the normal flow rate, such as 10%, 15%, 20%, or 25% of the normal flow rate. In one or more examples, the flow rate of blood that flows through the internal channel of the catheter 200 may be more than 50% of the normal flow rate, such as 70%, 80%, or 90% of the normal flow rate. The balloon 202 can be configured such that when it is fully inflated it contacts the body lumen and/or an occluded region of the body lumen. Alternatively, the balloon 202 may be configured to expand only a specified amount when fully inflated (such as expand in diameter by 50% relative to its uninflated diameter).

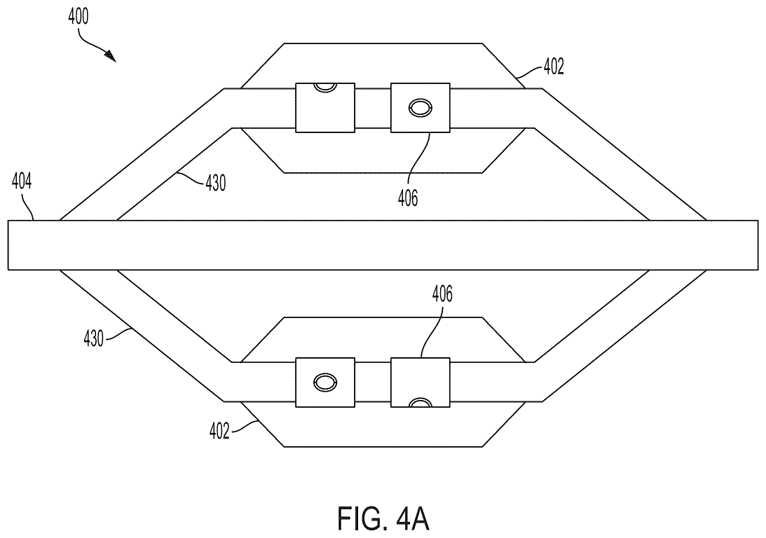

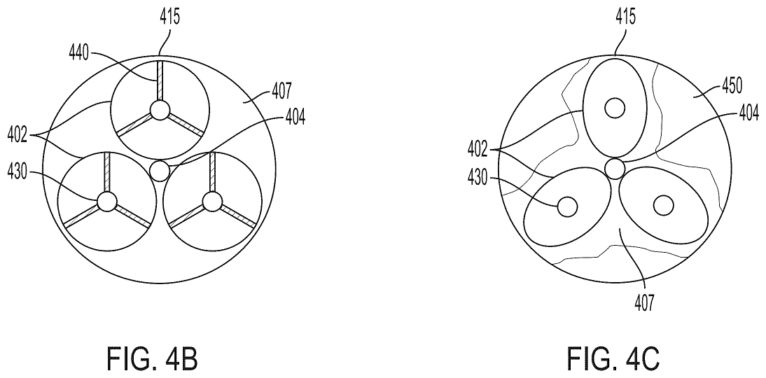

The balloon 202 is formed from a material having elastomeric properties such that the balloon can accept inflation pressures of up to approximately six atmospheres. In one or more examples, the balloon 202 may be compliant enough to accept inflation pressures of more than six atmospheres, such as inflation pressures up to ten atmospheres. The balloon 202 can be formed from a polymeric material currently used in the medical device industry. The balloon 202 can be configured to assume a circular shape (as viewed from a cross sectional perspective) while inflated. Optionally, the balloon 202 may be configured to assume an elliptical shape, or any other suitable shape. The balloon 202 may include structural support members (for instance as shown in FIG. 4B) such as framing struts or ribbing in the balloon material that cause the balloon 202 to assume a particular shape when inflated. Structural support members such as these may be collapsible such that they collapse around the elongated tube 204 when the balloon 202 is not filled, and may incorporate resilient features, such as a spring, that cause the structural support members to expand away from the elongated tube 204 as the balloon 202 is filled.

FIG. 2B illustrates a cross sectional view of an exemplary elongated tube 203 comprising an internal channel that is usable for a shock wave perfusion balloon catheter. The elongated tube 203 is a hollow tube that forms a single lumen 230. Accordingly, when blood flows through the elongated tube 203, (such as by entering and exiting via holes located proximally and distally relative to a balloon) blood flows through the hollow interior of the elongated tube 203. The elongated tube 203 has a diameter d₁ which may be, for example, between 0.066 inches (5 Fr.) and 0.1 inches (8 Fr.).

FIG. 2C illustrates a cross sectional view of an exemplary elongated tube 205 comprising a central guidewire lumen and an internal channel that is usable for a shock wave perfusion balloon catheter. In contrast to the elongated tube 203, the elongated tube 205 includes a central guidewire tube 209 defining a central guidewire lumen 290. Accordingly, the elongated tube 205 includes a pair of lumens: a central guidewire lumen 290, and an outer blood flow lumen 250, the internal channel that is defined by the annular lumen formed in the space between the elongated tube 205 and the central guidewire tube 209. When blood flows through the elongated tube 205 (such as by entering and exiting via holes located proximally and distally relative to a balloon) blood flows through the blood flow lumen 250. The elongated tube 205 has a diameter d₂ which may be the same as the diameter d₁ of the elongated tube 203. Alternatively, the diameter d₂ of the elongated tube 205 may be greater than the diameter d₁ of the elongated tube 203, for instance, between 0.1 inches (8 Fr.) and 0.13 inches (10 Fr.). The central guidewire tube 209 may have a diameter of approximately 0.015-0.016 inches such that the central guidewire tube 209 can accommodate a guidewire that is approximately 0.014 inches in diameter. In one or more examples, the central guidewire tube 209 may be sized to receive a guidewire that is up to 0.035 inches in diameter.

The elongated tube 203 of FIG. 2B or the elongated tube 205 of FIG. 2C may be used as the elongated tube 204 of catheter 200 of FIG. 2A. When the catheter 200 includes the elongated tube 203 of FIG. 2B, a guidewire may initially be inserted within the interior of the elongated tube 203 in order to position the balloon 202 proximate to a calcification in a body lumen and then retracted prior to beginning treatment. That is, a guidewire may be retracted prior to filling the balloon 202 with conductive fluid and applying voltage to the shock wave generators 206 to generate shock waves. The guidewire may be retracted from the catheter 200 completely (e.g., removed outside the body), or may be retracted only partially (e.g., retracted to a location that is proximal of the proximal holes 208 such that the guidewire is not located in the channel that blood flows through to bypass the balloon 202).

The elongated tube 204 may optionally include a barrier 211 located distally of the distal holes 210 that helps to cause blood to exit the internal channel via the distal holes 210. In addition or alternatively, the elongated tube 204 may include a barrier 211 located proximally of the proximal holes 208 that helps to cause blood to exit the internal channel via the proximal holes 208, depending on the direction of blood flow through the body lumen. The barrier 211 may be, for example, a dispensing valve with a central opening that permits a guidewire to pass through, an umbrella seal, a sealing member, a check valve, etc.

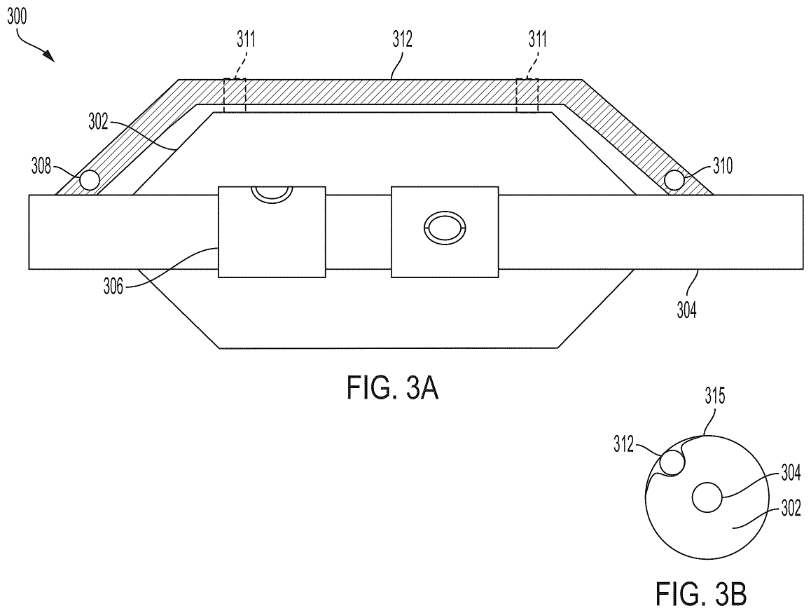

FIG. 3A illustrates the distal end of an exemplary shock wave perfusion balloon catheter 300 that has at least one external lumen 312 (which can alternatively be referred to as a bypass lumen). The catheter 300 includes an elongated tube 304 and a balloon 302 sealed to the elongated tube 304. The elongated tube 304 can include a hollow interior that receives a guidewire. The balloon 302 surrounds a pair of shock wave generators 306, which are circumferentially wrapped around the elongated tube 304. In one or more examples, the balloon 302 can surround more than or less than two shock wave generators 306. Each of the shock wave generators includes at least one pair of electrodes, with the electrodes of each electrode pair spaced apart from one another to form a spark gap. The balloon 302 is fillable with conductive fluid, such as saline, such that the balloon 302 expands to provide an annular channel between an inner surface of the balloon 302 and the shock wave generators 306.

The catheter 300 can be used for catheter 100 in system 10 of FIG. 1 and can be connected to a pulsed power source such as the power source 150 to supply voltage to the shock wave generators 306 within the balloon 302 to generate shock waves at the spark gaps between the electrode pairs of the shock wave generators 306. The catheter 300 is illustrated with a pair of shock wave generators 306. However, this is provided for explanatory purposes only, and the catheter 300 may have a single shock wave generator 306 or have more than two shock wave generators 306 within the balloon 302.

The catheter 300 is similar to the catheter 200 of FIG. 2A except that instead of an internal channel within the elongated tube 304, the catheter 300 includes at least one external lumen 312. The external lumen 312 is located along the outer surface of the balloon 302, and is attached to the elongated tube 304 at a location that is proximal of the proximal end of the balloon 302 and at a location that is distal of the distal end of the balloon 302 such that the external lumen 312 acts as a bypass channel around the balloon 302. The external lumen 312 can be attached to the elongated tube 304 in a sealed manner at each of these locations. The external lumen 312 includes at least one proximal hole 308 located on a proximal end of the external lumen 312 and at least one distal hole 310 located on a distal end of the external lumen 312.

When the balloon 302 is located in a body lumen and filled with fluid (i.e., when inflated), blood in the body lumen can enter the external lumen 312 via the proximal hole(s) 308, travel through the external lumen 312, and exit the external lumen 312 via the distal hole(s) 310. If blood is flowing in the opposite direction, blood may enter the external lumen 312 via the distal hole(s) 310 and exit the external lumen 312 via the proximal hole(s) 308. FIG. 3B illustrates a cross sectional view of the distal end of the shock wave perfusion balloon catheter 300 in a body lumen 315. When inflated, the balloon 302 expands to fill the space within the body lumen 315 but does not fill the space occupied by external lumen 312. Thus, blood can continue to flow through the body lumen 315 through the internal lumen 312 while the balloon 302 is inflated. Accordingly, the external lumen 312 provides a channel that permits blood to flow through the body lumen past the balloon 302 while the balloon 302 is inflated. In one or more examples, the catheter 300 can include multiple external lumens 312.

The flow rate of the blood that flows through the external lumen 312 of the catheter 300 when the balloon 302 is fully inflated can be approximately 50% of the flow rate of blood that would normally flow through the body lumen without the catheter 300 placed in the body lumen (i.e., the “normal flow rate”). In one or more examples, the flow rate of blood that flows through the external lumen 312 of the catheter 300 may be less than 50% of the normal flow rate, such as 10%, 15%, 20%, or 25% of the normal flow rate. In one or more examples, the flow rate of blood that flows through the external lumen 312 of the catheter 300 may be more than 50% of the normal flow rate, such as 70%, 80%, or 90% of the normal flow rate. The balloon 302 can be configured such that when it is fully inflated it contacts the body lumen and/or an occluded region of the body lumen. Alternatively, the balloon 302 may be configured to expand only a specified amount when fully inflated (such as expand in diameter by 50% relative to its uninflated diameter).

The external lumen 312 can be formed from a semi-compliant material that permits the external lumen 312 to flex outwardly when the balloon 302 is inflated. For instance, the external lumen 312 can be formed from polyurethane, nylon, polyethylene (PE), polyolefin copolymer (POC), polyethylene terephthalate (PET), or any combination thereof. The external lumen 312 can be located between the balloon 302 and a calcification when the catheter 300 is used to treat a calcification in a body lumen. Thus, when voltage is applied to the shock wave generators 306 (such as via wires as shown in FIG. 1) and shock waves begin propagating outwards, the shock waves can travel through the balloon 302 and through the external lumen 312 before travelling into the calcification. The material of the outer surface of the external lumen 312 may thus be relatively strong such that contact with calcification in the body lumen does not damage the external lumen 312. In the expanded configuration, the balloon 302 may not be under pressure. Accordingly, the pressure that presses the external lumen 312 against a calcification may not be sufficient to buckle the external lumen 312 (e.g., flatten the channel). The external lumen 312 may be detachable such that the external lumen 312 can be removed and a new external lumen 312 can be attached to the catheter 300. The external lumen 312 may be fixedly attached to an outer surface of the balloon 302 such as via one or more seals 311.

A shock wave catheter as described herein may include multiple balloons. For example, FIG. 4A illustrates the distal end of an exemplary tri-balloon shock wave catheter 400. The catheter 400 includes a central elongated tube 404, which can include a hollow interior that receives a guidewire. Three side tubes 430 (two are shown in the view depicted in FIG. 4A) are attached to the elongated tube 404, extending outwardly. A balloon 402 is sealed to each of the side tubes 430. The tri-balloon shock wave catheter 400 includes three side tubes 430, one for each balloon 402. This is provided for example only, as a multi-balloon shock wave catheter as described herein could include a variety of multi-balloon configurations such as a quad-balloon catheter containing four side tubes and four balloons, a bi-balloon catheter containing two side tubes and two balloons, etc.

Each balloon 402 surrounds a pair of shock wave generators 406, which are circumferentially wrapped around one of the side tubes 430. In one or more examples, each balloon 402 can surround more than or less than two shock wave generators 406. Each shock wave generator 406 includes at least one pair of electrodes, with the electrodes of each electrode pair spaced apart from one another to form a spark gap. The balloons 402 are fillable with conductive fluid, such as saline, such that the balloons 402 expand to provide an annular channel between an inner surface of the balloon 402 and each shock wave generator 406.

The catheter 400 can be used for catheter 100 in system 10 of FIG. 1 and can be connected to a pulsed power source such as the power source 150 to supply voltage to the shock wave generators 406 within each balloon 402 to generate shock waves at the spark gaps between the electrode pairs of the shock wave generators 406. As shown in FIG. 4A, each balloon 402 surrounds a pair of shock wave generators 406. However, this is provided for explanatory purposes only, and the catheter 400 may be configured to include a single shock wave generator 406 within each balloon 402, more than two shock wave generators 406 within each balloon 402, or to include different numbers of shock wave generators 406 within different balloons 402 (e.g., one shock wave generator 406 in a first balloon and two shock wave generators 406 in a second balloon). The balloons 402 of the catheter 400 may be a number of shapes. For instance, the balloons 402 may be circular or elliptical when viewed from a cross sectional perspective. The balloons 402 may be configured to assume the circular or elliptical shape based on manufacturing steps during the balloon-forming process.

The side tubes 430 can be connected to the elongated tube 404 in a sealed manner. When the catheter 400 is located in a body lumen and the balloons 402 are filled with fluid (i.e., when inflated), the side tubes 430 extend outwardly from the central elongated tube 404, thereby creating a channel (or multiple channels) in the space between the balloons 402. Accordingly, blood can flow through the channel(s) defined by the space between the balloons 402 and thus may flow through the body lumen past the balloons 402 while the balloons 402 are inflated. The channel(s) between the balloons 402 are more clearly visible in a cross-sectional view of the catheter 400. FIG. 4B illustrates a cross sectional view of the tri-lobe balloon shock wave catheter 400 comprising circular balloons 402, and FIG. 4C illustrates a cross sectional view of the tri-lobe balloon shock wave catheter 400 comprising elliptical balloons 402.

The views shown in FIGS. 4B and 4C depict the catheter 400 within a body lumen 415. As shown in FIG. 4B, at least one channel 407 extends between the balloons 402, providing space for blood to flow past the balloons 402 of the catheter 400 during treatment. Similarly, as shown in FIG. 4C, which depicts the catheter 400 within an occluded body lumen 415, the at least one channel 407 extends between the occluded areas 450 of the body lumen 415 and the balloons 402 such that blood can continue to flow through the body lumen 415 during treatment. As noted above, the balloons of a shock wave catheter such as the catheter 400 can include structural support members (e.g., ribs, struts, scaffolding, springs, shape-memory material, etc.) that cause the balloon to assume a particular shape when inflated. Exemplary structural support members 440 are shown in the balloons 402 of the embodiment depicted in FIG. 4B. These structural support members 440 can expand away from the side tubes 430 as the balloons 402 are inflated to cause the balloons 402 to assume the circular shape shown. When the balloons 402 are deflated, the structural support members 440 can collapse around the side tubes 430.

When the balloons 402 are filled with fluid, the overall diameter of the catheter 400 can be, for example, between 0.066 inches and 0.1 inches (5-8 Fr.). In one or more examples, the overall diameter of the catheter 400 can be greater, such as between 0.1 inches and 0.197 inches (8-15 Fr.). The catheter 400 may be suitable for treating a wide range of body lumens, such as the aorta, aortic valve, peripheral arteries, etc.

The flow rate of the blood that flows through the channel(s) between the balloons 402 of the catheter 400 when the balloons 402 are fully inflated can be approximately 50% of the flow rate of blood that would normally flow through the body lumen without the catheter 400 placed in the body lumen (i.e., the “normal flow rate”). In one or more examples, the flow rate of blood that flows through the channel(s) between the balloons 402 of the catheter 400 may be less than 50% of the normal flow rate, such as 10%, 15%, 20%, or 25% of the normal flow rate. In one or more examples, the flow rate of blood that flows through the channel(s) between the balloons 402 of the catheter 400 may be more than 50% of the normal flow rate, such as 70%, 80%, or 90% of the normal flow rate. The catheter 400 can be configured such that when the balloons 402 are fully inflated, they contact the body lumen and/or an occluded region of the body lumen. Alternatively, the balloons 402 may be configured to expand only a specified amount when fully inflated (such as expand in diameter by 50% relative to their uninflated diameter).

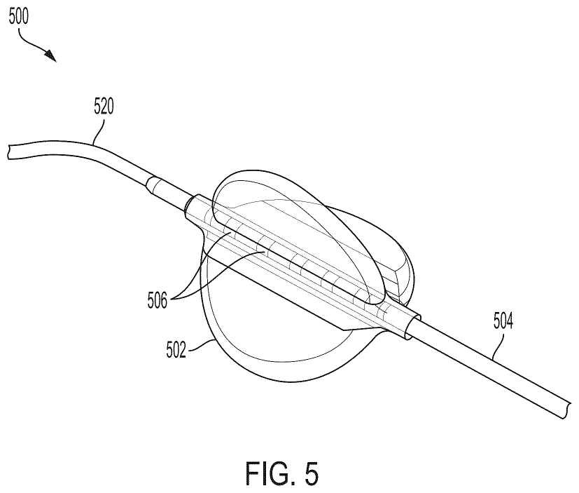

As shown in FIG. 4A, the balloons 402 of the catheter 400 are entirely separate from one another, as each is sealed to a separate side tube 430 of the catheter 400. In one or more examples, a multi-lobe balloon catheter may instead incorporate a single balloon that is sealed to the central elongated tube 404 and includes multiple lobes. An example of this is shown in FIG. 5, which illustrates the distal end of an exemplary tri-lobe balloon shock wave catheter 500 comprising a central elongated tube 504. The catheter 500 includes a tri-lobe balloon 502 that is sealed to the elongated tube 504. A plurality of shock wave generators 506 are circumferentially wrapped around the elongated tube 504 within the tri-lobe balloon 502. The elongated tube 504 includes a hollow interior that receives the guidewire 520, which is shown protruding from the distal end of the elongated tube 504.

The catheter 500 can be used for catheter 100 in system 10 of FIG. 1 and can be connected to a pulsed power source such as the power source 150 to supply voltage to the shock wave generators 506 within the tri-lobe balloon 502 to generate shock waves at the spark gaps between the electrode pairs of the shock wave generators 506. As shown in FIG. 5, the shock wave generators 506 are located on the elongated tube 504, and do not extend within the lobes of the tri-lobe balloon 502. When generating shock waves from the shock wave generators 506, shock waves can propagate generally outwardly. In one or more examples, the shock wave generators 506 may be oriented to propagate shock waves primarily along the direction of the lobes of the tri-lobe balloon 502, such that the shock waves propagate through the conductive fluid within the lobes. Alternatively or in addition, the shock wave generators 506 may be oriented to propagate shock waves primarily in the space between the lobes of the tri-lobe balloon 502. In one or more examples, the catheter 500 can include side tubes (similar to the side tubes 430 of FIG. 4A) that extend into the lobes of the balloon 502 and include additional shock wave generators. Accordingly, the catheter 500 can include shock wave generators 506 dispersed throughout the lobes of the balloon 502, rather than located only in the center of the balloon 502 surrounding the elongated tube 504.

The lobes of the tri-lobe balloon 502 extend outwardly from the elongated tube 504 when filled with fluid (i.e., when inflated). When deflated, the lobes of the tri-lobe balloon 502 may be positioned closely proximate to the elongated tube 504, which improves the maneuverability of the catheter 500. Once inflated, the lobes of the tri-lobe balloon 502 may contact a body lumen and act to hold the catheter 500 in place within a calcified area of the body lumen (e.g., as shown in FIG. 4C). Channel(s) are defined by the spaces between the lobes of the tri-lobe balloon 502, which provide space for blood to flow through a body lumen past the lobes of the tri-lobe balloon 502 when the tri-lobe balloon 502 is inflated. Compared to the catheter 400 of FIG. 4A, the channel(s) extending between the lobes of the tri-lobe balloon 502 may be larger, and thus may provide for a greater flow rate. The tri-lobe balloon 502 is provided for example only, as a catheter according to the invention disclosed herein may include a balloon with a variety of multi-lobe configurations such as a quad-lobe balloon with four lobes, a d₁-lobe balloon with two lobes, etc.

The flow rate of the blood that flows through the channel(s) between the lobes of the tri-lobe balloon 502 of the catheter 500 when the tri-lobe balloon 502 is fully inflated can be approximately 50% of the flow rate of blood that would normally flow through the body lumen without the catheter 500 placed in the body lumen (i.e., the “normal flow rate”). In one or more examples, the flow rate of blood that flows through the channel(s) between the lobes of the tri-lobe balloon 502 of the catheter 500 may be less than 50% of the normal flow rate, such as 10%, 15%, 20%, or 25% of the normal flow rate. In one or more examples, the flow rate of blood that flows through the channel(s) between the lobes of the tri-lobe balloon 502 of the catheter 500 may be more than 50% of the normal flow rate, such as 70%, 80%, or 90% of the normal flow rate. The tri-lobe balloon 502 can be configured such that when it is fully inflated the lobes contact the body lumen and/or an occluded region of the body lumen. Alternatively, the tri-lobe balloon 502 may be configured to expand only a specified amount when fully inflated (such as expand in diameter by 50% relative to its uninflated diameter).

As noted above, a multi-balloon shock wave catheter as described herein could include a variety of multi-balloon configurations and is not limited to a tri-balloon design. For example, a quad-balloon shock wave catheter could include four side tubes and four balloons, a quint-balloon shock wave catheter could include five side tubes and five balloons, a sext-balloon shock wave catheter could include six side tubes and six balloons, etc. Additionally, a multi-balloon shock wave catheter may incorporate balloons having different sizes relative to one another. For instance, the three balloons 402 of the tri-balloon shock wave catheter 400 could each be different sizes and/or shapes (e.g., a first circular balloon, a second elliptical balloon, and a third circular balloon having a different diameter from the first circular balloon).

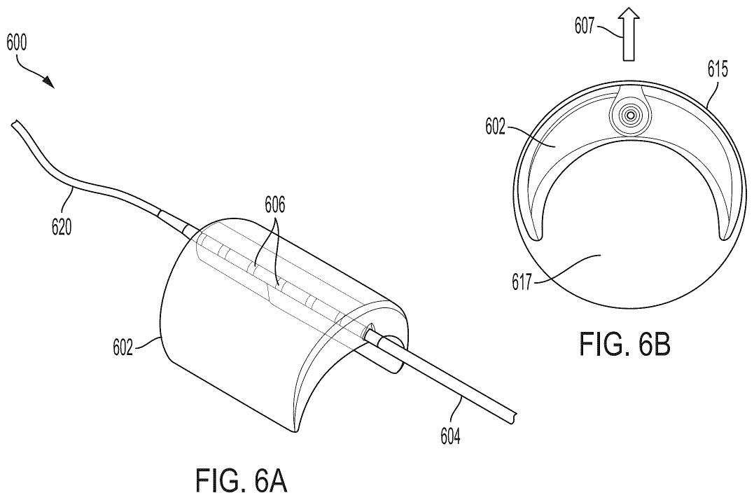

FIG. 6A illustrates the distal end of a crescent-shaped balloon shock wave catheter 600. The catheter 600 includes a crescent-shaped balloon 602 that is sealed to an elongated tube 604. A plurality of shock wave generators 606 are circumferentially wrapped around the elongated tube 604 within the balloon 602. The elongated tube 604 includes a hollow interior that receives the guidewire 620, which is shown protruding from the distal end of the elongated tube 604.

The catheter 600 can be used for catheter 100 in system 10 of FIG. 1 and can be connected to a pulsed power source such as the power source 150 to supply voltage to the shock wave generators 606 within the balloon 602 to generate shock waves at the spark gaps between the electrode pairs of the shock wave generators 606. As shown in FIG. 6A, the shock wave generators 606 are positioned in the center of the balloon 602. In one or more examples, the catheter 600 can include side tubes (similar to the side tubes 430 of FIG. 4A) that extend into the peaks of the crescent (e.g., outwardly from the center of the balloon) and include additional shock wave generators. Accordingly, the catheter 600 can include shock wave generators 606 dispersed throughout the balloon, rather than located only in the center of the balloon 602 surrounding the elongated tube 604.

When generating shock waves from the shock wave generators 606, shock waves can propagate generally outwardly. In one or more examples, the shock wave generators 606 may be oriented to propagate shock waves generally upwardly with respect to the view shown in in FIG. 6B (as shown by arrow 607 of FIG. 6B). Alternatively or in addition, the shock wave generators 606 may be oriented to propagate shock waves in a variety of directions outwardly from the elongated tube 604.

FIG. 6B illustrates a cross sectional view of the catheter 600 of FIG. 6A positioned within a body lumen 615. When the balloon 602 is filled with fluid (i.e., when inflated), the balloon 602 assumes a crescent-shape. The balloon 602 can be configured and/or molded to assume the crescent-shape when inflated based on manufacturing steps during the balloon-forming process. The balloon 602 may include internal structural features that ensure the balloon 602 will assume the crescent-shape when inflated (e.g., ribs, struts, scaffolding, springs, shape-memory material, etc.). When deflated, the balloon 602 may be positioned closely proximate to the elongated tube 604, which improves the maneuverability of the catheter 600. Once inflated, the crescent-shape of the balloon 602 leaves an opening 617 (that is defined by the crescent-shape of the balloon 602), which provides a channel that permits blood to flow through the body lumen 615 past the balloon 602 while the balloon 602 is inflated.

The flow rate of the blood that flows through the channel (i.e., the opening 617) of the catheter 600 when the balloon 602 is fully inflated can be approximately 50% of the flow rate of blood that would normally flow through the body lumen without the catheter 600 placed in the body lumen (i.e., the “normal flow rate”). In one or more examples, the flow rate of blood that flows through the channel of the balloon 602 may be less than 50% of the normal flow rate, such as 10%, 15%, 20%, or 25% of the normal flow rate. In one or more examples, the flow rate of blood that flows through the channel of the balloon 602 may be more than 50% of the normal flow rate, such as 70%, 80%, or 90% of the normal flow rate. The balloon 602 can be configured such that when it is fully inflated it contacts the body lumen and/or an occluded region of the body lumen. Alternatively, the balloon 602 may be configured to expand only a specified amount when fully inflated (such as expand in diameter by 50% relative to its uninflated diameter).

As shown in FIG. 6B, the balloon 602 contacts only a portion of the walls of the body lumen 615 (e.g., only the top portion). When in use, a surgeon may treat portions of the body lumen 615 sequentially, such as by rotating the elongate tube 604 to cause the balloon 602 to rotate within the body lumen 615 and contact a new portion of the walls of the body lumen 615. For instance, the surgeon may incrementally rotate the elongated tube 604 in a clockwise direction to treat the entirety of the body lumen 615. Beneficially, to treat the entirety of the body lumen 615 using the catheter 600, the surgeon would not have to deflate and re-inflate the balloon 602, as may be necessary when relying on a balloon that does not include a channel to permit blood to flow past the catheter during treatment.

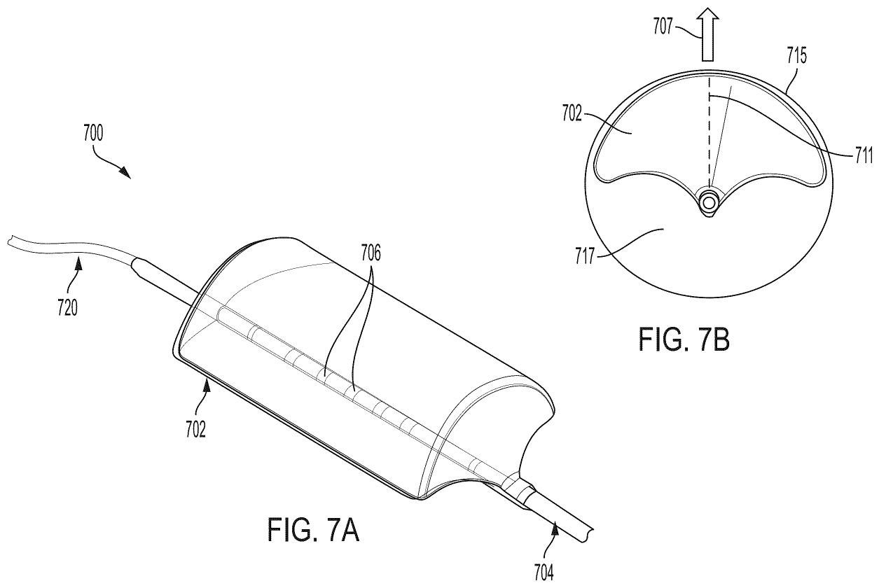

FIG. 7A illustrates the distal end of a double crescent-shaped balloon shock wave catheter 700. The catheter 700 includes a double crescent-shaped balloon 702 that is sealed to an elongated tube 704. As shown more clearly in FIG. 7B which illustrates a cross sectional view of the catheter 700 of FIG. 7A positioned within a body lumen 715, when the balloon 702 is filled with fluid (e.g., inflated), the balloon 702 has two concave regions (e.g., two “crescents”). That is, when the balloon 702 is filled with fluid, the balloon 702 assumes a double crescent-shape. A plurality of shock wave generators 706 are circumferentially wrapped around the elongated tube 704 within the balloon 702. The elongated tube 704 includes a hollow interior that receives the guidewire 720, which is shown protruding from the distal end of the elongated tube 704.

The catheter 700 can be used for catheter 100 in system 10 of FIG. 1 and can be connected to a pulsed power source such as the power source 150 to supply voltage to the shock wave generators 706 within the balloon 702 to generate shock waves at the spark gaps between the electrode pairs of the shock wave generators 706. As shown in FIG. 7A, the shock wave generators 706 are positioned in the center of the balloon 702. In one or more examples, the catheter 700 can include side tubes (similar to the side tubes 430 of FIG. 4A) that extend into the peaks of the crescent (e.g., outwardly from the center of the balloon) and include additional shock wave generators. Accordingly, the catheter 700 can include shock wave generators 706 dispersed throughout the balloon, rather than located only in the center of the balloon 702 surrounding the elongated tube 704.

When generating shock waves from the shock wave generators 706, shock waves can propagate generally outwardly. In one or more examples, the shock wave generators 706 may be oriented to propagate shock waves generally upwardly with respect to the view shown in in FIG. 7B (as shown by arrow 707 of FIG. 7B). Alternatively or in addition, the shock wave generators 706 may be oriented to generate shock waves in a variety of directions outwardly from the elongated tube 704.

The balloon 702 can be configured and/or molded to assume the double crescent-shape when inflated based on manufacturing steps during the balloon-forming process (e.g., ribs, struts, scaffolding, springs, shape-memory material, etc.). The balloon 702 may include internal structural features that ensure the balloon 702 will assume the double crescent-shape when inflated. For example, the balloon 702 can include an internal wall 711 that ensures the balloon 702 assumes the double crescent-shape when inflated. The internal wall 711 can be a feature included in the balloon 702 during the balloon-forming process. When deflated, the balloon 702 may be positioned closely proximate to the elongated tube 704, which improves the maneuverability of the catheter 700. Once inflated, the double crescent-shape of the balloon 702 leaves an opening 717, which provides a channel that permits blood to flow through the body lumen 715 past the catheter 700 while the balloon 702 is inflated.

The flow rate of the blood that flows through the channel (i.e., the opening 717) of the catheter 700 when the balloon 702 is fully inflated can be approximately 50% of the flow rate of blood that would normally flow through the body lumen without the catheter 700 placed in the body lumen (i.e., the “normal flow rate”). In one or more examples, the flow rate of blood that flows through the channel of the balloon 702 may be less than 50% of the normal flow rate, such as 10%, 15%, 20%, or 25% of the normal flow rate. In one or more examples, the flow rate of blood that flows through the channel of the balloon 702 may be more than 50% of the normal flow rate, such as 70%, 80%, or 90% of the normal flow rate. The balloon 702 can be configured such that when it is fully inflated it contacts the body lumen and/or an occluded region of the body lumen. Alternatively, the balloon 702 may be configured to expand only a specified amount when fully inflated (such as expand in diameter by 50% relative to its uninflated diameter).

As shown in FIG. 7B, the balloon 702 contacts only a portion of the walls of the body lumen 715 (e.g., the top portion). When in use, a surgeon may treat portions of the body lumen 715 sequentially, such as by rotating the elongated tube 704 to cause the balloon 702 to rotate within the body lumen 715 and contact a new portion of the walls of the body lumen 715. For instance, the surgeon may incrementally rotate the elongated tube 704 in a clockwise direction to treat the entirety of the body lumen 715. Beneficially, to treat the entirety of the body lumen 715 using the catheter 700, the surgeon would not have to deflate and re-inflate the balloon 702, as may be necessary when relying on a balloon that does not include a channel to permit blood to flow past the catheter during treatment.

Although the electrode assemblies and catheter devices described herein have been discussed primarily in the context of treating coronary occlusions, such as lesions in vasculature, the electrode assemblies and catheters herein can be used for a variety of occlusions, such as occlusions in the peripheral vasculature (e.g., above-the-knee, below-the-knee, iliac, carotid, etc.). For further examples, similar designs may be used for treating soft tissues, such as cancer and tumors (i.e., non-thermal ablation methods), blood clots, fibroids, cysts, organs, scar and fibrotic tissue removal, or other tissue destruction and removal. Electrode assembly and catheter designs could also be used for neurostimulation treatments, targeted drug delivery, treatments of tumors in body lumens (e.g., tumors in blood vessels, the esophagus, intestines, stomach, or vagina), wound treatment, non-surgical removal and destruction of tissue, or used in place of thermal treatments or cauterization for venous insufficiency and fallopian ligation (i.e., for permanent female contraception).

In one or more examples, the electrode assemblies and catheters described herein could also be used for tissue engineering methods, for instance, for mechanical tissue decellularization to create a bioactive scaffold in which new cells (e.g., exogenous or endogenous cells) can replace the old cells; introducing porosity to a site to improve cellular retention, cellular infiltration/migration, and diffusion of nutrients and signaling molecules to promote angiogenesis, cellular proliferation, and tissue regeneration similar to cell replacement therapy. Such tissue engineering methods may be useful for treating ischemic heart disease, fibrotic liver, fibrotic bowel, and traumatic spinal cord injury (SCI). For instance, for the treatment of spinal cord injury, the devices and assemblies described herein could facilitate the removal of scarred spinal cord tissue, which acts like a barrier for neuronal reconnection, before the injection of an anti-inflammatory hydrogel loaded with lentivirus to genetically engineer the spinal cord neurons to regenerate.

The elements and features of the exemplary electrode assemblies and catheters discussed above may be rearranged, recombined, and modified, without departing from the present invention. Furthermore, numerical designators such as “first”, “second”, “third”, “fourth”, etc. are merely descriptive and do not indicate a relative order, location, or identity of elements or features described by the designators. For instance, a “first” shock wave may be immediately succeeded by a “third” shock wave, which is then succeeded by a “second” shock wave. As another example, a “third” emitter may be used to generate a “first” shock wave and vice versa. Accordingly, numerical designators of various elements and features are not intended to limit the disclosure and may be modified and interchanged without departing from the subject invention.

It should be noted that the elements and features of the example catheters illustrated throughout this specification and drawings may be rearranged, recombined, and modified without departing from the present invention. For instance, while this specification and drawings describe and illustrate catheters having several example balloon designs, the present disclosure is intended to include catheters having a variety of balloon configurations. The number, placement, and spacing of the electrode pairs of the shock wave generators can be modified without departing from the subject invention. Further, the number, placement, and spacing of balloons of catheters can be modified without departing from the subject invention.

It should be understood that the foregoing is only illustrative of the principles of the invention, and that various modifications, alterations and combinations can be made by those skilled in the art without departing from the scope and spirit of the invention. Any of the variations of the various catheters disclosed herein can include features described by any other catheters or combination of catheters herein. Furthermore, any of the methods can be used with any of the catheters disclosed. Accordingly, it is not intended that the invention be limited, except as by the appended claims.

1. A catheter for treating a stenosis in a body lumen, the catheter comprising:
an elongated tube;
at least one shock wave generator comprising at least one electrode pair;
at least one balloon sealed to a distal end of the elongated tube and surrounding the at least one shock wave generator, the at least one balloon fillable with a conductive fluid; and
at least one channel that permits blood to flow through the body lumen past the at least one balloon while the at least one balloon is inflated.

2. The catheter of claim 1, wherein the at least one channel permits blood to flow through the body lumen at a flow rate that is at least 50% of a normal flow rate of blood through the body lumen without the catheter positioned in the body lumen.

3. The catheter of claim 1, wherein the at least one channel is defined by at least one lumen that extends externally to the elongated tube.

4. The catheter of claim 3, wherein the at least one lumen extends outside of an outer surface of the at least one balloon.

5. The catheter of claim 1, wherein the at least one channel extends within the elongated tube.

6. The catheter of claim 5, wherein the elongated tube comprises at least one opening to the at least one channel that is located proximally of the at least one balloon for blood to flow into or out of the at least one channel, and at least one opening to the at least one channel from the at least one channel that is located distally of the at least one balloon for blood to flow into or out of the at least one channel.

7. The catheter of claim 1, wherein the catheter comprises a plurality of balloons and a plurality of shock wave generators, each balloon of the plurality of balloons being sealed to a region of the elongated tube and surrounding one or more of the plurality of shock wave generators, wherein the at least one channel is defined by separation between the plurality of balloons.

8. The catheter of claim 7, wherein the plurality of balloons is at least three balloons.

9. The catheter of claim 8, wherein a cross section of each of the at least three balloons comprises a circular shape.

10. The catheter of claim 1, wherein a cross section of each of the at least three balloons comprises an elliptical shape.

11. The catheter of claim 1, wherein the at least one balloon comprises a plurality of lobes that extend outwardly from the elongated tube, and wherein the at least one channel is defined by separation between the plurality of lobes of the at least one balloon.

12. The catheter of claim 1, wherein the at least one balloon comprises a crescent shape when inflated, and the at least one channel is defined by space between the at least one balloon and the body lumen.

13. The catheter of claim 1, wherein the at least one balloon comprises a double crescent shape when inflated, and the at least one channel is defined by space between the at least one balloon and the body lumen.

14. The catheter of claim 1, wherein the elongated tube comprises a guidewire lumen for receiving a guidewire, and wherein the catheter is configured to be advanced into the body lumen over the guidewire.

15. A system for treating a stenosis in a body lumen comprising:
a catheter comprising:
an elongated tube;
at least one shock wave generator comprising at least one electrode pair;
at least one balloon sealed to a distal end of the elongated tube and surrounding the at least one shock wave generator, the at least one balloon fillable with a conductive fluid; and
at least one channel that permits blood to flow through the body lumen past the at least one balloon while the at least one balloon is inflated; and
a power source configured to apply a voltage pulse to the at least one shock wave generator to generate shock waves for treating the stenosis.

16. The system of claim 15, wherein the at least one channel permits blood to flow through the body lumen at a flow rate that is at least 50% of a normal flow rate of blood through the body lumen without the catheter positioned in the body lumen.

17. The system of claim 15, wherein the at least one channel is defined by at least one lumen that extends externally to the elongated tube.

18. The system of claim 17, wherein the at least one lumen extends outside of an outer surface of the at least one balloon.

19. The system of claim 15, wherein the at least one channel extends within the elongated tube.

20. The system of claim 19, wherein the elongated tube comprises at least one opening to the at least one channel that is located proximally of the at least one balloon for blood to flow into or out of the at least one channel, and at least one opening to the at least one channel from the at least one channel that is located distally of the at least one balloon for blood to flow into or out of the at least one channel.

21. The system of claim 15, wherein the catheter comprises a plurality of balloons and a plurality of shock wave generators, each balloon of the plurality of balloons being sealed to a region of the elongated tube and surrounding one or more of the plurality of shock wave generators, wherein the at least one channel extends between the plurality of balloons.

22. The system of claim 21, wherein the plurality of balloons is at least three balloons.

23. The system of claim 22, wherein a cross section of each of the at least three balloons comprises a circular shape.

24. The system of claim 22, wherein a cross section of each of the at least three balloons comprises a circular shape.

25. The system of claim 15, wherein the at least one balloon comprises a plurality of lobes that extend outwardly from the elongated tube, and wherein the at least one channel is defined by separation between the plurality of lobes of the at least one balloon.

26. The system of claim 15, wherein the at least one balloon comprises a crescent shape when inflated, and wherein the at least one channel is defined by separation between the plurality of lobes of the at least one balloon.

27. The system of claim 15, wherein the at least one balloon comprises a double crescent shape when inflated, and wherein the at least one channel is defined by separation between the plurality of lobes of the at least one balloon.

28. The system of claim 15, wherein the elongated tube comprises a guidewire lumen for receiving a guidewire, and wherein the catheter is configured to be advanced into the body lumen over the guidewire.

29. A method for treating a stenosis in a body lumen, the method comprising:
advancing a catheter within the body lumen to a position proximate to the stenosis;
inflating at least one balloon of the catheter so that an outer surface of the at least one balloon contacts the body lumen and blood is permitted to flow through the body lumen past the at least one balloon while the at least one balloon is inflated; and
generating shock waves via at least one shock wave generator of the catheter while blood is flowing through the body lumen past the at least one balloon.

30. The method of claim 29, comprising advancing a guidewire within the body lumen to locate the stenosis and advancing the catheter over the guidewire.

31. The method of claim 30, comprising retracting the guidewire before inflating the balloon.

32. The method of claim 31, comprising retracting the guidewire such that a distal end of the guidewire is located proximally of a proximal end of the balloon.
